SEQUENCE LISTING (38 sequences)

SEQ ID NO: 1 - DNA, 201 bp, Beta vulgaris
acacargggacgaaaagcagaacakgacaccaacacartagcaataacgacacaacaagagcagcaacataacaacaattcaatgacccactaacaataaaccaccacamctccttttagctaaactcacaatcactaatcaatctgctaattcaaatcttgaartgaaataaaatcyatttctragtaaactcagcaaac

SEQ ID NO: 2 - DNA, 201 bp, Beta vulgaris
acacargggacgaaaagcagaacakgacaccaacacartagcaataacgacacaacaagagcagcaacataacaacaattcaatgacccactaacaataagccaccacamctccttttagctaaactcacaatcactaatcaatctgctaattcaaatcttgaartgaaataaaatcyatttctragtaaactcagcaaac

SEQ ID NO: 3 - DNA, 201 bp, Beta vulgaris
tactcaagcacgtaaatggttcaacgaaatggttctggatggctgttcccctaatgtggtgacctacactgccctaattcatgcataccttaaagtacgctggataggtgaagccaatgaactttttgagattatgttgaaagatggatgcaaaccgaatgttgtgacttatactgctttaattgatggttattgtaaatc

SEQ ID NO: 4 - DNA, 201 bp, Beta vulgaris
tactcaagcacgtaaatggttcaacgaaatggttctggatggctgttcccctaatgtggtgacctacactgccctaattcatgcataccttaaagtacgcaggataggtgaagccaatgaactttttgagattatgttgaaagatggatgcaaaccgaatgttgtgacttatactgctttaattgatggttattgtaaatc

SEQ ID NO: 5 - DNA, 201 bp, Beta vulgaris
gagctgatccggtttgtttcaaaccataagcctattgttgctgctatgagggtgtctgagagaactgtcatgattatcaagaacctagtatcgtcttcggtgccatctctggtatggtagtattgacatatagaaaatgccaatttttcagttcttggtcatattcacttaaggacagtatgcttgtacagtcttctctat

SEQ ID NO: 6 - DNA, 201 bp, Beta vulgaris
gagctgatccggtttgtttcaaaccataagcctattgttgctgctatgagggtgtctgagagaactgtcatgattatcaagaacctagtatcgtcttcggcgccatctctggtatggtagtattgacatatagaaaatgccaatttttcagttcttggtcatattcacttaaggacagtatgcttgtacagtcttctctat

SEQ ID NO: 7 - DNA, 201 bp, Beta vulgaris
caaagtaagaagtcagaaaatttatattcctagcctctcttcctcaccccttttcctgtatgttgtgtgagctttgattagttttaacgaatataattctttcgctgcaaacctgcaagtgtgtatatactccattttgtatatatatgaaatcatggagtattgcagtgaggaatctttgtaagactttctctgagaatg

SEQ ID NO: 8 - DNA, 201 bp, Beta vulgaris
caaagtaagaagtcagaaaatttatattcctagcctctcttcctcaccccttttcctgtatgttgtgtgagctttgattagttttaacgaatataattctgtcgctgcaaacctgcaagtgtgtatatactccattttgtatatatatgaaatcatggagtattgcagtgaggaatctttgtaagactttctctgagaatg

SEQ ID NO: 9 - DNA, 201 bp, Beta vulgaris
tttcagaatcttcagcttcatggatagaaccaatgtgttgagtattcatcgcttgtcgattgataactgaccaacttgtctttcgagtttgcgggagttggactggagggcccaaggttacaggtttaagggcttgtgcagctaaggttttcatgatgaagcagaacaatctctggcagattccaagagattgaagctgct

SEQ ID NO: 10 - DNA, 201 bp, Beta vulgaris
tttcagaatcttcagcttcatggatagaaccaatgtgttgagtattcatcgcttgtcgattgataactgaccaacttgtctttcgagtttgcgggagttgtactggagggcccaaggttacaggtttaagggcttgtgcagctaaggttttcatgatgaagcagaacaatctctggcagattccaagagattgaagctgct

SEQ ID NO: 11 - DNA, 201 bp, Beta vulgaris
  misc_feature (178)..(178): n is a, c, g, or t
ccaacaacttrtttctccaactcttcctcttagcttccatatycatgccttgtgccaaatccattaatttcaactatcctgcagttttcaactttggcgactcgaattctgataccggtgctcttgtggcttctggactcgaggggatyactgatccctacggacaracttacttcanaaaaccatcaggaagatacagtg

SEQ ID NO: 12 - DNA, 201 bp, Beta vulgaris
  misc_feature (178)..(178): n is a, c, g, or t
ccaacaacttrtttctccaactcttcctcttagcttccatatycatgccttgtgccaaatccattaatttcaactatcctgcagttttcaactttggcgattcgaattctgataccggtgctcttgtggcttctggactcgaggggatyactgatccctacggacaracttacttcanaaaaccatcaggaagatacagtg

SEQ ID NO: 13 - DNA, 201 bp, Beta vulgaris; identical to SEQ ID NO: 12
  misc_feature (178)..(178): n is a, c, g, or t

SEQ ID NO: 14 - DNA, 201 bp, Beta vulgaris
ttacttttgccaagaatatactttgtttgcatggttcaacgtaaagttgattggtgcgagaartgttatattttctggtktgggccaccagaaacgyagcgcttttccacakwacactaaatggatcctttctggacctggcaaaactaggttgtacacccaaataactctatataggtaggaatttactgtattccaaat

SEQ ID NO: 15 - DNA, 201 bp, Beta vulgaris
  misc_feature (103)..(103): n is a, c, g, or t
  misc_feature (151)..(151): n is a, c, g, or t
aacggttggaatttttctgaagacattactgttttgtaattgtccatgaaggtgactagcttcgggaatggtcattttaggtgcaagcgatgatcttgttatntgagtggtcaatcttgatggaaagaaatgcttagggatggtgtttgtntgctaagaatggagaagaaaccaataagctaagatcagttcatgtctcgt

SEQ ID NO: 16 - DNA, 201 bp, Beta vulgaris
  misc_feature (103)..(103): n is a, c, g, or t
  misc_feature (151)..(151): n is a, c, g, or t
aacggttggaatttttctgaagacattactgttttgtaattgtccatgaaggtgactagcttcgggaatggtcattttaggtgcaagcgatgatcttgttctntgagtggtcaatcttgatggaaagaaatgcttagggatggtgtttgtntgctaagaatggagaagaaaccaataagctaagatcagttcatgtctcgt

SEQ ID NO: 17 - DNA, 201 bp, Beta vulgaris
  misc_feature (182)..(182): n is a, c, g, or t
ctattagttttgatgtgtcattttatggtgctgattttgtttatggtattcctgaacatgctactagtcttgcacttaagcctactagaggccccggtatcgaacattcggaaccttataggctttttaacttggatgtgtttgaatatcttcatgagtcgcctttcgggttgtatggatcnattccgttcatgcttggtc

SEQ ID NO: 18 - DNA, 201 bp, Beta vulgaris
  misc_feature (182)..(182): n is a, c, g, or t
ctattagttttgatgtgtcattttatggtgctgattttgtttatggtattcctgaacatgctactagtcttgcacttaagcctactagaggccccggtattgaacattcggaaccttataggctttttaacttggatgtgtttgaatatcttcatgagtcgcctttcgggttgtatggatcnattccgttcatgcttggtc

SEQ ID NO: 19 - DNA, 201 bp, Beta vulgaris
tgcgccaaattatcaacctgtggtgcatgaacttatacatgaaaaaacaaaacgaactcatacatgaaaaccaaaagaggtggaccctaaatgtaataacaaacctggtgagtgagccacatgagcaagatatcaacagcagggaccaagacacttgaagaagcatcaccatcaacatccccatcatcttgttggattacc

SEQ ID NO: 20 - DNA, 201 bp, Beta vulgaris
tgcgccaaattatcaacctgtggtgcatgaacttatacatgaaaaaacaaaacgaactcatacatgaaaaccaaaagaggtggaccctaaatgtaataactaacctggtgagtgagccacatgagcaagatatcaacagcagggaccaagacacttgaagaagcatcaccatcaacatccccatcatcttgttggattacc

SEQ ID NO: 21 - DNA, 201 bp, Beta vulgaris
agaatgtgaggggaagaaacmgaagatgcattgcatgaagaaggatgragtataatagaaccccaatccctctctccatacttggcctctttcagtcttctccttactaacaatcctcccacatcttcttttctcatctcagaaacactcctcagtattgcaaggcayaataacagcacgaaaacagcrtcttcttctggc

SEQ ID NO: 22 - DNA, 201 bp, Beta vulgaris
agaatgtgaggggaagaaacmgaagatgcattgcatgaagaaggatgragtataatagaaccccaatccctctctccatacttggcctctttcagtcttcgccttactaacaatcctcccacatcttcttttctcatctcagaaacactcctcagtattgcaaggcayaataacagcacgaaaacagcrtcttcttctggc

SEQ ID NO: 23 - DNA, 201 bp, Beta vulgaris
aaagaaatactttgacatggtggcgtaartattttcccgtgcttagactgataaatccaacatcaattctattatcatgaaaaagcaaatttacctctttggatatttgctgcagaataggtatgatctcagcgaagtcaggtctcattgcaggatcttgctgccagcatctttcaagaagctcggtaagcttgggatgac

SEQ ID NO: 24 - DNA, 201 bp, Beta vulgaris
aaagaaatactttgacatggtggcgtaartattttcccgtgcttagactgataaatccaacatcaattctattatcatgaaaaagcaaatttacctcttttgatatttgctgcagaataggtatgatctcagcgaagtcaggtctcattgcaggatcttgctgccagcatctttcaagaagctcggtaagcttgggatgac

SEQ ID NO: 25 - DNA, 201 bp, Beta vulgaris
aagcagatccttaacttcccttcttaactgacgcagttttgagtcttaagaatgacaaccctatgctttcatttggatattcaatgtctactgaagccactactacaggcttgattactgttaaatttgtaccaaaatgggtaaattacagtttattgttctactgataatgatttttgtaaacattcaagtgctttgtat

SEQ ID NO: 26 - DNA, 201 bp, Beta vulgaris
aagcagatccttaacttcccttcttaactgacgcagttttgagtcttaagaatgacaaccctatgctttcatttggatattcaatgtctactgaagccaccactacaggcttgattactgttaaatttgtaccaaaatgggtaaattacagtttattgttctactgataatgatttttgtaaacattcaagtgctttgtat

SEQ ID NO: 27 - DNA, 201 bp, Beta vulgaris
caaaagactataagggtcaagttgaagcaaacttgagagtggtcctccactggatcatttgtctctattgtcttaatgcaacaaacgacttgaaaccatctaaaaacaatagataggatgattccactagaatcatcattgcgaataagcaaccaaggatctttattgtttaccacataaacattcatattgatcaccatg

SEQ ID NO: 28 - DNA, 201 bp, Beta vulgaris
caaaagactataagggtcaagttgaagcaaacttgagagtggtcctccactggatcatttgtctctattgtcttaatgcaacaaacgacttgaaaccatccaaaaacaatagataggatgattccactagaatcatcattgcgaataagcaaccaaggatctttattgtttaccacataaacattcatattgatcaccatg

SEQ ID NO: 29 - DNA, 201 bp, Beta vulgaris
atatcattgaaaaaataatattgaggaaataaagttcaacctgtttagcacctagaccaaaggttgcattgccagatagataaggtttcgagcgatcagcggttttgcagagcatagaaagtgctgcagtagctcccacttgtatagcctgcatgtcctccaaaatggtgagaagctgaatagcttaagattcataattct

SEQ ID NO: 30 - DNA, 201 bp, Beta vulgaris
atatcattgaaaaaataatattgaggaaataaagttcaacctgtttagcacctagaccaaaggttgcattgccagatagataaggtttcgagcgatcagcagttttgcagagcatagaaagtgctgcagtagctcccacttgtatagcctgcatgtcctccaaaatggtgagaagctgaatagcttaagattcataattct

SEQ ID NO: 31 - DNA, 201 bp, Beta vulgaris
aggtaatatataaaacagaggcaatctcacaataaaacagttatcatggsgatcagattcacaaacccgtaacagcaacatgagggtcaaaacagccaccagataaacttcaaaaagcccgacccgaccccaactactgggcagcaacagccaacttaaagcttaccagcccaagctacgaycaatcaacatkgtaacatc

SEQ ID NO: 32 - DNA, 201 bp, Beta vulgaris
aggtaatatataaaacagaggcaatctcacaataaaacagttatcatggsgatcagattcacaaacccgtaacagcaacatgagggtcaaaacagccaccggataaacttcaaaaagcccgacccgaccccaactactgggcagcaacagccaacttaaagcttaccagcccaagctacgaycaatcaacatkgtaacatc

SEQ ID NO: 33 - DNA, 201 bp, Beta vulgaris
gacaccacctaatcaaataatgctttcttcattagtagggaaccctatccaagaacaagaacttacctcaagtgattttcgaggtgaggggctacgagagattccagcttccccacgcggagaaacygcctgrggagaagccaaacgatgaggaattggtgcwcgttcatcraggccgygcttctctgggctacgactcct

SEQ ID NO: 34 - DNA, 201 bp, Beta vulgaris
gacaccacctaatcaaataatgctttcttcattagtagggaaccctatccaagaacaagaacttacctcaagtgattttcgaggtgaggggctacgagaggttccagcttccccacgcggagaaacygcctgrggagaagccaaacgatgaggaattggtgcwcgttcatcraggccgygcttctctgggctacgactcct

SEQ ID NO: 35 - DNA, 201 bp, Beta vulgaris
aagaattctttgtgttaaggtggttgtgtgatgtatgtttgatagtagcaaagattcctttgatatatgtagattaatatagtcaatttttatctatcatctgtagtagcactctcaagttcatctgatattcgtgtaataattctgaacgcttgaatgcattggttgtttttgtttagacattaaactcttttgttcagg

SEQ ID NO: 36 - DNA, 201 bp, Beta vulgaris
aagaattctttgtgttaaggtggttgtgtgatgtatgtttgatagtagcaaagattcctttgatatatgtagattaatatagtcaatttttatctatcatttgtagtagcactctcaagttcatctgatattcgtgtaataattctgaacgcttgaatgcattggttgtttttgtttagacattaaactcttttgttcagg

SEQ ID NO: 37 - DNA, 201 bp, Beta vulgaris
aacttatattacatgagtgacattcatataggtcacttacgcgaacatctggtagattatcaatgcctaaatttgaagtatccttctcatattcaacaggtccacatgaattacttatagttgcaagggaaaggtccagcgtggagttgccatcctcagaattcacgcacatatgctgttagatagcagcatacaaatgtt

SEQ ID NO: 38 - DNA, 201 bp, Beta vulgaris
aacttatattacatgagtgacattcatataggtcacttacgcgaacatctggtagattatcaatgcctaaatttgaagtatccttctcatattcaacaggcccacatgaattacttatagttgcaagggaaaggtccagcgtggagttgccatcctcagaattcacgcacatatgctgttagatagcagcatacaaatgtt

(19) United States
(12) Patent Application Publication
(10) Pub. No.: US 2024/0251730 A1
(43) Pub. Date: Aug. 1, 2024

(54) Plants of the Species Beta Vulgaris With Resistance to Cercospora

(71) Applicant: Bejo Zaden B.V., Warmenhuizen (NL)

(72) Inventors: Jacob van Dorp, Warmenhuizen (NL); Rosaline Anna Maria Jong, Warmenhuizen (NL); Dora Lisa Moita E Coelho, Warmenhuizen (NL); Albertus Johannes Maria Schrijver, Warmenhuizen (NL)

(21) Appl. No.: 18/563,957

(22) PCT Filed: May 28, 2021
(86) PCT No.: PCT/EP2021/064365

§ 371 Date: Nov. 24, 2023

(51) Int. Cl. A01H 1/00; A01H 5/06; A01H 6/02; C12Q 1/6895

(52) U.S. Cl. CPC A01H 1/1255; A01H 5/06; A01H 6/024; C12Q 1/6895

(57) ABSTRACT

Provided herein are Beta vulgaris plants resistant to Cercospora. Also provided herein are methods for identifying Cercospora resistant Beta vulgaris plants, methods for providing Cercospora resistant Beta vulgaris plants and means for identifying Cercospora resistant Beta vulgaris plants. Specifically, provided herein are Cercospora-resistant Beta vulgaris plants including a first Cercospora resistance providing genomic fragment on chromosome 4, wherein said first Cercospora resistance providing genomic fragment includes at least one sequence from the group consisting of SEQ ID Nos. 1, 3, 5, 7, 9, 11, 13, and 15.

### CROSS-REFERENCE TO RELATED APPLICATION

This application is the United States national phase of International Application No. PCT/EP2021/064365 filed May 28, 2021, the disclosure of which is hereby incorporated by reference in its entirety.

The Sequence Listing associated with this application is filed in electronic format via Patent Center and is hereby incorporated by reference into the specification in its entirety. The name of the text file containing the Sequence Listing is 2307809_ST25.txt. The size of the text file is 15,557 bytes, and the text file was created on Oct. 18, 2023.

### BACKGROUND OF THE INVENTION

### Field of the Invention

The present invention relates to Beta vulgaris plants resistant to Cercospora. The present invention further relates to methods for identifying Cercospora resistant Beta vulgaris plants, methods for providing Cercospora resistant Beta vulgaris plants and means for identifying Cercospora resistant Beta vulgaris plants.

### Description of Related Art

Beta vulgaris plants belong to the subfamily Betoideae of the family Amaranthaceae. Other members of this family are the species Beta corolliflora, Beta lomatogona, Beta macrocarpa, Beta macrorhiza, Beta nana, and Beta trigyna. A plant of the species Beta vulgaris is, in particular, a plant of the subspecies Beta vulgaris subsp. vulgaris. This subspecies comprises several cultivar groups, such as beetroot (Beta vulgaris ssp. vulgaris var. conditiva; alternatively referred to as Beta vulgaris ssp. vulgaris var. rubra), also known as garden beet, red beet or table beet), grown as a root vegetable; sugar beet (Beta vulgaris ssp. vulgaris var. altissima), cultivated for the production of sugar; stalky chard, or Swiss chard (Beta vulgaris subsp. vulgaris var. flavescens), and leafy spinach beet (Beta vulgaris subsp. vulgaris var. cicla), grown for their leaves; and mangelwurzel (Beta vulgaris ssp. vulgaris var. crassa), which is also known as fodder beet, or mangold wurzel, and is a fodder crop. Genomically encoded traits, such as disease resistance, in any of these cultivar groups can be introduced into another cultivar via human action using methods known to a person skilled in the art.

Cultivated Beta vulgaris plants are grown worldwide, generally in regions with temperate climates. The plant prefers relatively cool temperatures between 15 and 24° C. Globally, Europe, especially France and Germany, the United States of America and Russia are the largest producers of beets. However, beet cultivars are also grown in many other countries in Europe, Asia, Africa and South America.

Cercospora Leaf Spot (CLS), caused by the ascomycete fungus Cercospora beticola, is considered one of the most economically damaging diseases that affect plants of the species Beta vulgaris due to its worldwide prevalence and destructiveness. Damage to the plant foliage caused by this disease can, for sugarbeets, decrease yield up to 50% and, for beetroots, reduce marketability and interfere with mechanized harvesting.

Cercospora leaf spot occurs globally in regions where beets are cultivated, resulting in significant damage specially in warm temperate areas, including southern Europe, the Mediterranean region, Japan, Russia, USA and South and Central America.

Infection occurs rapidly at temperatures 25-35° C., in particular when night temperatures are above 16° C. and the relative humidity is above >90%, and is further promoted by rainy weather and leaf irrigation.

The infection starts when a spore lands on a leaf and, with its germ tubes, penetrates the leaf through stomata. The germ tube then develops into hyphae that grow intercellularly inside the leaf. The hyphae produce toxins, such as cercosporin and beticolin, and other enzymes that damage and kill nearby plant cells. This necrosis causes the appearance of round, millimeter-size, brown spots (3-5 mm) on leaves, the first visible sign of infection. As the disease progresses, these brown spots grow and merge until the leave itself turns yellow and dies. Cercospora can defoliate an entire plant requiring it to grow new leaves, which costs energy that cannot be directed to the beet.

Cercospora spreads by forming specialized hyphae (conidiophores) that emerge from the leaf and form asexual spores called conidia. These conidia are disseminated by wind or rain. Under suitable conditions, these conidia can germinate and repeat the cycle of infection. Cercospora leaf spot is a polycyclic disease characterized by repeated cycles of conidia germination, infection, sporulation, conidia release and re-infection. Under favorable conditions, one cycle of infection and re-infection can be completed within ten days leading to multiple infection cycles during the growing season and rapid spread of the infection to adjacent leaves and plants.

Outside growing seasons, Cercospora can survive on plant debris as conidia (for 1-4 months) and pseudostromata, where it can persist for 1-2 years. Alternatively, the fungus can infect weed species related to beets (e.g., lamb's quarters and pigweed) as well as other vegetable crops (chard, spinach).

Measures to manage cercospora leaf spot mainly include fungicidal treatments and crop rotation. However, limiting the use of fungicidal measures is highly desired as they affect the environment and are expensive. Moreover, chemical control with fungicides has become increasingly difficult due to the spread of various strains of Cercospora that are resistant to important chemical classes of fungicides. More efficient and sustainable disease management, therefore, requires the development of varieties with genetic resistance.

Currently, all commercial beetroot varieties are susceptible to cercospora leaf spot despite Cercospora-tolerant varieties of sugar beet being on the market for decades. All of these tolerant varieties contain the same resistance originating from Beta vulgaris spp. maritima, which has a number of drawbacks:
The resistance is quantitative and based on an unknown number of QTLs leading to complicated inheritance patterns. Previous publications identified at least four or five causative QTLs on chromosomes 2, 3, 4, 6 and 9 of the beet genome. Each of these QTLs only had a small contribution to resistance (less than 5%).
The resistance is linked to reduced agronomic performance in sugar beet. One or several of the resistance genes may be flanked by a gene or genes coding for poor agronomic performances. This is called linkage drag. Alternatively, the large number of QTLs limits effective inbreeding of Cercospora-resistant parent lines resulting in reduced hybrid vigour.
The resistance is not sufficient to protect the crop against cercospora leaf spot. Fungicide applications are nevertheless still needed.

Recently, a novel Cercospora-resistance gene in plants of the species Beta vulgaris, in particular sugarbeets, was described (EP3696188). The advantage of this new resistance is that it is based on a single gene, and, in sugar beet, it does not have a negative effect on agronomic performance. Yet, the resistance provided by this gene is limited and additional fungicide treatment or further genetic resistance is still required to protect the crop against cercospora leaf spot.

Hence, to generate beet varieties with improved resistance against cercospora leaf spot, there is a need to identify new, preferably augmented, genomically encoded resistances, which on their own provide a strong resistance effect without reducing agronomic performance. These resistances can then be stacked to generate beet varieties, especially commercial hybrid beetroot varieties, with strong and durable resistance against Cercospora beticola.

### SUMMARY OF THE INVENTION

Considering the above, it is an object of the present invention, amongst others, to provide novel genomically encoded resistances to Cercospora beticola and plants comprising these resistances. The present invention especially has the object, amongst other objects, to provide a beetroot plant resistant to Cercospora beticola.

The present invention meets the above object, amongst other objects, as outlined in the appended claims.

Specifically, this object, amongst other objects, is achieved by providing Cercospora-resistant Beta vulgaris plants comprising a genomic fragment located on chromosome 4, wherein said genomic fragment comprises at least one sequence from the group consisting of SEQ ID No. 1. SEQ ID No. 3. SEQ ID No. 5, SEQ ID No. 7, SEQ ID No. 9, SEQ ID No. 11, SEQ ID No. 13, and SEQ ID No. 15.

### DESCRIPTION OF THE INVENTION

The term “at least one” can be used interchangeably with the term “one or more”. As such, the term “at least one” should be understood to mean at least one, two, three, four, five, six, or more, or all. Preferably, the resistance providing genomic fragment on chromosome 4 comprises SEQ ID No. 5; more preferably SEQ ID No. 5, and SEQ ID No. 7; more preferably SEQ ID No. 3, SEQ ID No. 5, and SEQ ID No. 7; more preferably SEQ ID No. 1. SEQ ID No. 3. SEQ ID No. 5, SEQ ID No. 7, and SEQ ID No. 9; and most preferably SEQ ID No. 1. SEQ ID No. 3. SEQ ID No. 5, SEQ ID No. 7, SEQ ID No. 9, SEQ ID No. 11, SEQ ID No. 13, and SEQ ID No. 15. The resistance providing genomic fragment is not comprised of SEQ ID No. 2. SEQ ID No. 4, SEQ ID No. 6, SEQ, ID No. 8, SEQ ID No. 10, SEQ ID No. 12, SEQ ID No. 14, and SEQ ID No. 16. Said resistance providing genomic fragment on chromosome 4 may further comprise at least one sequence from the group consisting of SEQ ID No. 17, SEQ ID No. 19, and SEQ ID No. 21.

Alternatively, this object, amongst other objects, is achieved by providing Cercospora-resistant Beta vulgaris plants comprising a combination of two resistance providing genomic fragments, wherein a first genomic fragment is located on chromosome 4 and comprises at least one sequence from the group consisting of SEQ ID No. 1. SEQ ID No. 3, SEQ ID No. 5, SEQ ID No. 7, SEQ ID No. 9. SEQ ID No. 11, SEQ ID No. 13, and SEQ ID No. 15, and wherein, a second genomic fragment is located on chromosome 9 and comprises at least one sequence from the group consisting of SEQ ID No. 23, SEQ ID No. 25, SEQ ID No. 27, SEQ ID No. 29, SEQ ID No. 31, SEQ ID No. 33, SEQ ID No. 35, and SEQ ID No. 37. Preferably, said first genomic fragment on chromosome 4 comprises SEQ ID No. 5; more preferably SEQ ID No. 5, and SEQ ID No. 7; more preferably SEQ ID No. 3, SEQ ID No. 5, and SEQ ID No. 7; more preferably SEQ ID No. 1. SEQ ID No. 3. SEQ ID No. 5, SEQ ID No. 7, and SEQ ID No. 9; and most preferably SEQ ID No. 1. SEQ ID No. 3, SEQ ID No. 5, SEQ ID No. 7, SEQ ID No. 9, SEQ ID No. 11, SEQ ID No. 13, and SEQ ID No. 15. Preferably, said second resistance providing genomic fragment on chromosome 9 comprises SEQ ID No. 35; more preferably SEQ ID No. 35, and SEQ ID No. 37; more preferably SEQ ID No. 33, SEQ ID No. 35, and SEQ ID No. 37; more preferably SEQ ID No. 29, SEQ ID No. 31, SEQ ID No. 33, SEQ ID No. 35, and SEQ ID No. 37; and most preferably SEQ ID No. 23, SEQ ID No. 25, SEQ ID No. 27, SEQ ID No. 29, SEQ ID No. 31, SEQ ID No. 33, SEQ ID No. 35, and SEQ ID No. 37. The first resistance providing genomic fragment is not comprised of SEQ ID No. 2, SEQ ID No. 4, SEQ ID No. 6, SEQ, ID No. 8, SEQ ID No. 10, SEQ ID No. 12, SEQ ID No. 14, and SEQ ID No. 16, and the second resistance providing genomic fragment is not comprised of SEQ ID No. 24, SEQ ID No. 26, SEQ ID No. 28, SEQ ID No. 30, SEQ ID No. 32, SEQ ID No. 34, SEQ ID No. 36, and SEQ ID No. 38. Hence, in one preferred embodiment, the Cercospora-resistant Beta vulgaris plant comprises SEQ ID No. 5, and SEQ ID No. 35; more preferably SEQ ID No. 1. SEQ ID No. 3. SEQ ID No. 5. SEQ ID No. 7, SEQ ID No. 9. SEQ ID No. 29, SEQ ID No. 31, SEQ ID No. 33, SEQ ID No. 35, and SEQ ID No. 37; and most preferably SEQ ID No. 1. SEQ ID No. 3. SEQ ID No. 5, SEQ ID No. 7, SEQ ID No. 9, SEQ ID No. 11, SEQ ID No. 13, SEQ ID No. 15, SEQ ID No. 23, SEQ ID No. 25, SEQ ID No. 27, SEQ ID No. 29, SEQ ID No. 31, SEQ ID No. 33, SEQ ID No. 35, and SEQ ID No. 37.

The resistance according to the invention can be introduced in a plant cell by transformation (e.g., using Agrobacterium tumefaciens). Genomic fragments can be amplified by long-range PCR amplifications, de novo synthesized, or isolated from gels or columns (e.g., after restriction digestion). The resulting fragments can be reassembled (e.g., in yeast) or introduced in an expression vector, subsequently transformed into Beta vulgaris cells and allowed to integrate or recombine with the Beta vulgaris genome. The fragment may be introduced in a single step or in a series of transformations ultimately resulting in a Beta vulgaris plant comprising the resistance of the present invention.

According to a preferred embodiment of the present invention, the resistance providing genomic fragments are obtainable, obtained, or are from a Beta vulgaris plant deposited at NCIMB (National Collections of Industrial, Food and Marine Bacteria; NCIMB Limited, Ferguson Building; Craibstone Estate, Bucksburn Aberdeen, Scotland, AB21 9YA United Kingdom) on 7 May 2021 under number NCIMB 43769.

According to a preferred embodiment, the present Beta vulgaris plants are cytoplasmic male sterile (CMS).

According to yet another preferred embodiment, the present Beta vulgaris plants are hybrid plants. The present invention also relates to hybrid Beta vulgaris plants obtainable either by crossing Cercospora-susceptible Beta vulgaris plants with Beta vulgaris plants comprising the resistance to Cercospora beticola or by crossing a Cercospora-susceptible Beta vulgaris plant with deposit number NCIMB 43769.

Within the context of the present invention the following Beta vulgaris plants are contemplated: Beta vulgaris ssp. vulgaris var. conditiva (beetroot, red beet), Beta vulgaris ssp. vulgaris var. altissima (sugar beet), Beta vulgaris subsp. vulgaris var. flavescens (chard, Swiss chard), Beta vulgaris subsp. vulgaris var. cicla (spinach beet), and Beta vulgaris ssp. vulgaris var. crassa (mangelwurzel).

The present invention further relates to methods for identifying the genomically encoded resistance against the plant pathogen Cercospora beticola as found in the Beta vulgaris plant deposited under deposit number NCIMB 43769. The method comprises the step of identifying one genomic fragment on chromosome 4 by detecting at least one genomic sequence from the group consisting of SEQ ID No. 1. SEQ ID No. 3, SEQ ID No. 5, SEQ ID No. 7, SEQ ID No. 9, SEQ ID No. 11, SEQ ID No. 13, SEQ ID No. 15, SEQ ID No. 17, SEQ ID No. 19, and SEQ ID No. 21. Preferably the method further comprises identifying a further resistance providing genomic fragment on chromosome 9 by detecting at least one genomic sequence from the group consisting of SEQ ID No. 23, SEQ ID No. 25, SEQ ID No. 27, SEQ ID No. 29, SEQ ID No. 31, SEQ ID No. 33, SEQ ID No. 35, and SEQ ID No. 37. Several common genotyping methods exist for detecting a single nuclear polymorphism (SNP) in a genomic sequence, including PCR-based methods, direct hybridization, fragment analysis, and sequencing. An example of a method suitable for detecting a genomic sequence is isolating DNA from available plant material (e.g., from a piece of a leaf from a plant, or a seed), followed by nucleic acid amplification of isolated DNA (e.g., using PCR), and detecting the presence of said genomic sequence (e.g., by sequencing, measuring fluorescence, or visualizing and analyzing PCR amplification using agarose gel electrophoresis).

The present invention accordingly provides a method of producing a Beta vulgaris plant of the invention comprising the steps of obtaining plant material from a plant of the species Beta vulgaris, subjecting said plant material to mutagenesis to create mutagenized plant material, analyzing said mutagenized plant material to identify a plant having resistance to Cercospora beticola. Said plant material is preferably a seed.

Suitable mutagenesis methods comprise chemical mutagenesis (e.g., using ethyl methanesulfonate (EMS), N-methyl-N-nitrosourea (MNU), N-ethyl-N-nitrosourea (ENU), sodium azide (NaN3), methylnitrosoguanidine (MNNG), diethyl sulfonate (DES), TILLING, or mutagenesis by generating reactive oxygen species) and radiation mutagenesis (e.g., using UV radiation or ion beam radiation). Mutagenesis can lead to one or more mutations located in the coding sequence (mRNA, cDNA or genomic sequence) or in the associated non-coding sequence and/or regulatory sequence regulating the level of expression of the coding sequence. The presence of one or more mutations (e.g., insertion, inversion, deletion and/or replacement of one or more nucleotide(s)) may lead to the encoded protein having a new or altered functionality (gain of function), reduced functionality (reduced function) or no functionality (loss-of-function), e.g., due to the protein being truncated or having an amino acid sequence wherein one or more amino acids are deleted, inserted or replaced. Such changes may lead to the protein having a different 3D structure or conformation, being targeted to a different sub-cellular compartment, having one or more modified catalytic domains, having a modified binding activity to nucleic acids or proteins, etc.

The present invention further relates to seeds capable of providing the present plants. Seeds can be coated, colored, washed, polished, encrusted, pelleted, primed or undergo a combination of treatments. Coated seeds are covered by a relatively thin layer of polymer supplied to the seed; to this polymer fungicides or insecticides can be added to protect the seed against soil borne pathogens and insect damage. Additionally, a dye can be added. This added color gives the farmer the opportunity to check for correct drilling of the seeds. Alternatively, also other beneficial compounds can be added as micronutrients or beneficial micro-organisms promoting the growth of the young seedlings. Encrusted seeds are not only covered by a polymer with or without extra substances, as described above, but the seeds are provided with a smooth surface as well. This makes drilling easier and the added weight enables a more precise direct drilling of the seeds. Polishing removes the outermost layer of the seed, so that the seed assumes a more rounded form. Polishing and washing promotes germination of the seed. With pelleting the seeds are covered with more material, e.g., polymer bound clay, to produce a regularly shaped, round pellet. This pellet, besides having protecting substances described above, can be constructed in such a way that it will melt or split after water uptake. Priming or pre-germination is a treatment in which seeds are given enough moisture to initiate germination of the embryo inside the seed. This results in a faster emergence of the seedling, a higher emergence rate and better growth. It is believed that priming leads to a better root system and faster growth.

In addition to the Beta vulgaris plant according to the invention, the present invention also relates to a seed, a plant cell, a protoplast, a plant organ, plant tissue, edible parts, pollen, microspores, ovaries, ovules, egg cells, callus, suspension culture, somatic embryos, embryos or plant parts of the Beta vulgaris plants defined above comprising a Cercospora beticola resistance providing genomic fragment. Plant parts include, but are not limited to, the shoot, the stalk, the stem, leaves, blossoms, inflorescence, roots, fruits, and cuttings.

The present invention furthermore relates to molecular markers which markers co-segregate with a genomically encoded resistance against the plant pathogen Cercospora beticola as present in deposit number NCIMB 43769, which molecular markers are selected from the group consisting of SEQ ID No. 1. SEQ ID No. 3, SEQ ID No. 5, SEQ ID No. 7, SEQ ID No. 9. SEQ ID No. 11, SEQ ID No. 13, SEQ ID No. 15, SEQ ID No. 17, SEQ ID No. 19, SEQ ID No. 21, SEQ ID No. 23, SEQ ID No. 25, SEQ ID No. 27, SEQ ID No. 29, SEQ ID No. 31, SEQ ID No. 33, SEQ ID No. 35, and SEQ ID No. 37.

The present invention will be further detailed in the following examples.

### Examples

### Example 1. Mutagenesis of Beta vulgaris Plants with the Purpose of Making a Library to Screen for Cercospora Resistance

Random mutagenesis followed by forward screening can be a useful method for identifying mutant Beta vulgaris plants with resistance to Cercospora beticola. A mutagenized library can be generated by subjecting seeds to a step of mutagenesis, preferably random mutagenesis. Such a step may comprise, but is not limited to, the treatment of a pool of 100.000 to 200.000 seeds with a chemical mutagen, or a mixture of chemical mutagens, e.g., 0.25% EMS for 16 hours at room temperature; alternatively, radiation can be used (e.g., gamma-radiation from a radioactive Cobalt-60 source). Preferably, only a mildly mutagenized library (fewer than 1% of all genes contain a mutation in a coding region) is generated. Nevertheless, the mutagenesis step will lead to the loss of germination in some seeds. In contrast to irradiation, which can lead to mutations varying from single base substitutions or deletions to large deletions, EMS produces predominantly random point mutations by nucleotide substitution; particularly by guanine alkylation.

The mutagenized seeds can be sown and propagated in a field using standard practices. The mutagenized seed will generate plants that each have a particular set of mutations. Plants can be harvested in pools and viable seeds can be sown again (F1 population). To obtain an F1S1 population seeds can be collected from the F1 plants after selfing. As the mutations will segregate in an F1S1 population, the resistance in this population can be evaluated and used to map the resistance against Cercospora beticola.

### Example 2. Field Tests for Assessing Resistance Against Cercospora beticola

The inoculum of Cercospora beticola was prepared by growing the fungus in Potato Dextrose Agar for 3 weeks incubated under near-UV light (12 hours day/12 hours night) at 25° C. Spores and mycelium were scraped with water, filtered and the concentration adjusted to 105 colony forming units per ml.

Plants were sown directly in the field in mid-June in the Netherlands, and, after germination, thinned to a 5 cm distance between plants. As susceptible control varieties Wodan F1 and Pablo F1 were used. Seven-week-old plants were inoculated by spraying the spore suspension on the leaves using an ultra-low volume or droplet sprayer, preferably during rainy weather or in the evening when dew will be formed during the night (as the fungus requires a long wet leaf period for infection). The inoculation was performed once a week, until a visibly high disease pressure was achieved (usually 8-12 consecutive weeks required, until mid-September to mid-October).

The assessment of disease resistance was performed visually by the amount and density of the typical small, round to oval-shaped spots on leaves, using a classification scale from 0 (completely susceptible, severe symptoms) to 9 (highly resistant, no symptoms). A score on this classification scale is referred to as a disease score.

### Example 3. Results of Assessment of Resistance Against Cercospora beticola

TABLE 1
Disease score of different genotypes and varieties

| | Disease Score Cercospora |
|---|---|
| | beticola inoculation test in NL |
| Susceptible varieties | 1-2 |
| (namely Wodan and Pablo) | |
| Plants comprising CB4.1 | 4 |
| Plants comprising CB4.1 + CB9.1 | 5 |

### Example 4. Development of Markers to Detect Cercospora Resistance in Beta vulgaris

A segregating F1S1 mapping population was developed to map the resistance against Cercospora beticola. This population was made by crossing a resistant Beta vulgaris plant with a susceptible Beta vulgaris plant, after which the resulting F1 plant was self-pollinated. To perform a QTL mapping, 520 individual plants were tested in a Cercospora beticola disease field trial and leaf material of these plants was used for DNA isolation, genotyping and marker analysis.

To genotype the mapping population, informative genome-wide markers were developed. Using 399 SNP markers covering the entire genome a genetic map was constructed and QTL analysis performed. Two QTL peaks were found. One QTL peak was located on chromosome 4 at base pair 45501459 of the EL10.1 public sugar beet reference genome (EL10_1.0, GenBank assembly accession GCA_002917755.1), and one on chromosome 9 at base pair 24213487 of the EL10.1 reference genome. The QTLs were named CB4.1 and CB9.1.

To narrow down the required haplotype size, a fine mapping experiment was performed. After pre-selection on 3720 plants, phenotypic assessment for the level of resistance was performed on 962 plants in a Cercospora beticola field disease trial. The results showed that CB4.1 is essential for resistance, and that CB9.1 is needed to obtain the highest level of resistance as combining the two QTL peaks gives the highest average disease score. SNPs for detecting the genomically encoded resistances against Cercospora beticola can be found in Table 2 to 4. Abbreviations are according to IUPAC nucleotide code.

Seeds of the F1S1 population comprising CB4.1 and CB9.1 were deposited at the NCIMB (NCIMB Limited, Ferguson Building; Craibstone Estate, Bucksburn ABERDEEN, Scotland, AB21 9YA, United Kingdom) on 7 May 2021 under deposit number NCIMB 43769.

| Symbol | Nucleotide Base |
|---|---|
| A | Adenine |
| C | Cytosine |
| G | Guanine |
| T | Thymine |
| N | A or C or G or T |
| M | A of C |
| R | A or G |
| W | A or T |
| S | C or G |
| Y | C or T |
| K | Gor T |
| V | Not T |
| H | Not G |
| D | Not C |
| B | Not A |

TABLE 2
SNPs for the detection of the resistance
against Cercospora beticola, QTL CB 4.1

| | | Position | Allele | |
|---|---|---|---|---|
| | Chromo- | Chromosome | linked | |
| | some | (bp) | to | Alternative |
| SNP | (EL10.1) | (EL10.1) | resistance | allele |
| 1 | 4 | 44276365 | A | G |
| 2 | 4 | 44440329 | T | A |
| 3 | 4 | 45501459 | T | C |
| 4 | 4 | 47197127 | T | G |
| 5 | 4 | 47371424 | G | T |
| 6 | 4 | 47419640 | C | T |
| 7 | 4 | 48257283 | A | G |
| 8 | 4 | 48338565 | A | C |
| 9 | 4 | 52795726 | C | T |
| 10 | 4 | 55593004 | A | T |
| 11 | 4 | 55597074 | T | G |

TABLE 3
SNPs for the detection of the resistance
against Cercospora beticola, QTL CB9.1

| | | Position | Allele | |
|---|---|---|---|---|
| | Chromo- | Chromosome | linked | |
| | some | (bp) | to | Alternative |
| SNP | (EL10.1) | (EL10.1) | resistance | allele |
| 12 | 9 | 21546628 | G | T |
| 13 | 9 | 21859451 | T | C |
| 14 | 9 | 22691063 | T | C |
| 15 | 9 | 23659048 | G | A |
| 16 | 9 | 24117497 | A | G |
| 17 | 9 | 24118744 | A | G |
| 18 | 9 | 24213487 | C | T |
| 19 | 9 | 24369620 | T | C |

TABLE 4
Sequences for the detection of the resistance
against Cercospora beticola

| SEQ | | SNP position in | |
|---|---|---|---|
| ID No. | SNP | EL10.1 | Sequence |
| 1 | 1 | Chr4_44276365 | ACACARGGGACGAAAAGCAGAACAKGACACCAAC |
| | | | ACARTAGCAATAACGACACAACAAGAGCAGCAAC |
| | | | ATAACAACAATTCAATGACCCACTAACAATAA[A]C |
| | | | CACCACAMCTCCTTTTAGCTAAACTCACAATCACT |
| | | | AATCAATCTGCTAATTCAAATCTTGAARTGAAATA |
| | | | AAATCYATTTCTRAGTAAACTCAGCAAAC |
| 2 | 1 | Chr4_44276365 | ACACARGGGACGAAAAGCAGAACAKGACACCAAC |
| | | | ACARTAGCAATAACGACACAACAAGAGCAGCAAC |
| | | | ATAACAACAATTCAATGACCCACTAACAATAA[G]C |
| | | | CACCACAMCTCCTTTTAGCTAAACTCACAATCACT |
| | | | AATCAATCTGCTAATTCAAATCTTGAARTGAAATA |
| | | | AAATCYATTTCTRAGTAAACTCAGCAAAC |
| 3 | 2 | Chr4_44440329 | TACTCAAGCACGTAAATGGTTCAACGAAATGGTTC |
| | | | TGGATGGCTGTTCCCCTAATGTGGTGACCTACACTG |
| | | | CCCTAATTCATGCATACCTTAAAGTACGC[T]GGATA |
| | | | GGTGAAGCCAATGAACTTTTTGAGATTATGTTGAA |
| | | | AGATGGATGCAAACCGAATGTTGTGACTTATACTG |
| | | | CTTTAATTGATGGTTATTGTAAATC |
| 4 | 2 | Chr4_44440329 | TACTCAAGCACGTAAATGGTTCAACGAAATGGTTC |
| | | | TGGATGGCTGTTCCCCTAATGTGGTGACCTACACTG |
| | | | CCCTAATTCATGCATACCTTAAAGTACGC[A]GGAT |
| | | | AGGTGAAGCCAATGAACTTTTTGAGATTATGTTGA |
| | | | AAGATGGATGCAAACCGAATGTTGTGACTTATACT |
| | | | GCTTTAATTGATGGTTATTGTAAATC |
| 5 | 3 | Chr4_45501459 | GAGCTGATCCGGTTTGTTTCAAACCATAAGCCTATT |
| | | | GTTGCTGCTATGAGGGTGTCTGAGAGAACTGTCAT |
| | | | GATTATCAAGAACCTAGTATCGTCTTCGG[T]GCCAT |
| | | | CTCTGGTATGGTAGTATTGACATATAGAAAATGCC |
| | | | AATTTTTCAGTTCTTGGTCATATTCACTTAAGGACA |
| | | | GTATGCTTGTACAGTCTTCTCTAT |
| 6 | 3 | Chr4_45501459 | GAGCTGATCCGGTTTGTTTCAAACCATAAGCCTATT |
| | | | GTTGCTGCTATGAGGGTGTCTGAGAGAACTGTCAT |
| | | | GATTATCAAGAACCTAGTATCGTCTTCGG[C]GCCAT |
| | | | CTCTGGTATGGTAGTATTGACATATAGAAAATGCC |
| | | | AATTTTTCAGTTCTTGGTCATATTCACTTAAGGACA |
| | | | GTATGCTTGTACAGTCTTCTCTAT |
| 7 | 4 | Chr4_47197127 | CAAAGTAAGAAGTCAGAAAATTTATATTCCTAGCC |
| | | | TCTCTTCCTCACCCCTTTTCCTGTATGTTGTGTGAGC |
| | | | TTTGATTAGTTTTAACGAATATAATTCT[T]TCGCTG |
| | | | CAAACCTGCAAGTGTGTATATACTCCATTTTGTATA |
| | | | TATATGAAATCATGGAGTATTGCAGTGAGGAATCT |
| | | | TTGTAAGACTTTCTCTGAGAATG |
| 8 | 4 | Chr4_47197127 | CAAAGTAAGAAGTCAGAAAATTTATATTCCTAGCC |
| | | | TCTCTTCCTCACCCCTTTTCCTGTATGTTGTGTGAGC |
| | | | TTTGATTAGTTTTAACGAATATAATTCT[G]TCGCTG |
| | | | CAAACCTGCAAGTGTGTATATACTCCATTTTGTATA |
| | | | TATATGAAATCATGGAGTATTGCAGTGAGGAATCT |
| | | | TTGTAAGACTTTCTCTGAGAATG |
| 9 | 5 | Chr4_47371424 | TTTCAGAATCTTCAGCTTCATGGATAGAACCAATGT |
| | | | GTTGAGTATTCATCGCTTGTCGATTGATAACTGACC |
| | | | AACTTGTCTTTCGAGTTTGCGGGAGTTG[G]ACTGGA |
| | | | GGGCCCAAGGTTACAGGTTTAAGGGCTTGTGCAGC |
| | | | TAAGGTTTTCATGATGAAGCAGAACAATCTCTGGC |
| | | | AGATTCCAAGAGATTGAAGCTGCT |
| 10 | 5 | Chr4_47371424 | TTTCAGAATCTTCAGCTTCATGGATAGAACCAATGT |
| | | | GTTGAGTATTCATCGCTTGTCGATTGATAACTGACC |
| | | | AACTTGTCTTTCGAGTTTGCGGGAGTTG[T]ACTGGA |
| | | | GGGCCCAAGGTTACAGGTTTAAGGGCTTGTGCAGC |
| | | | TAAGGTTTTCATGATGAAGCAGAACAATCTCTGGC |
| | | | AGATTCCAAGAGATTGAAGCTGCT |
| 11 | 6 | Chr4_47419640 | CCAACAACTTRTTTCTCCAACTCTTCCTCTTAGCTTC |
| | | | CATATYCATGCCTTGTGCCAAATCCATTAATTTCAA |
| | | | CTATCCTGCAGTTTTCAACTTTGGCGA[C]TCGAATT |
| | | | CTGATACCGGTGCTCTTGTGGCTTCTGGACTCGAGG |
| | | | GGATYACTGATCCCTACGGACARACTTACTTCANA |
| | | | AAACCATCAGGAAGATACAGTG |
| 12 | 6 | Chr4_47419640 | CCAACAACTTRTTTCTCCAACTCTTCCTCTTAGCTTC |
| | | | CATATYCATGCCTTGTGCCAAATCCATTAATTTCAA |
| | | | CTATCCTGCAGTTTTCAACTTTGGCGA[T]TCGAATT |
| | | | CTGATACCGGTGCTCTTGTGGCTTCTGGACTCGAGG |
| | | | GGATYACTGATCCCTACGGACARACTTACTTCANA |
| | | | AAACCATCAGGAAGATACAGTG |
| 13 | 7 | Chr4_48257283 | TTACTTTTGCCAAGAATATACTTTGTTTGCATGGTT |
| | | | CAACGTAAAGTTGATTGGTGCGAGAARTGTTATAT |
| | | | TTTCTGGTKTGGGCCACCAGAAACGYAGC[A]CTTTT |
| | | | CCACAKWACACTAAATGGATCCTTTCTGGACCTGG |
| | | | CAAAACTAGGTTGTACACCCAAATAACTCTATATA |
| | | | GGTAGGAATTTACTGTATTCCAAAT |
| 14 | 7 | Chr4_48257283 | TTACTTTTGCCAAGAATATACTTTGTTTGCATGGTT |
| | | | CAACGTAAAGTTGATTGGTGCGAGAARTGTTATAT |
| | | | TTTCTGGTKTGGGCCACCAGAAACGYAGC[G]CTTT |
| | | | TCCACAKWACACTAAATGGATCCTTTCTGGACCTG |
| | | | GCAAAACTAGGTTGTACACCCAAATAACTCTATAT |
| | | | AGGTAGGAATTTACTGTATTCCAAAT |
| 15 | 8 | Chr4_48338565 | AACGGTTGGAATTTTTCTGAAGACATTACTGTTTTG |
| | | | TAATTGTCCATGAAGGTGACTAGCTTCGGGAATGG |
| | | | TCATTTTAGGTGCAAGCGATGATCTTGTT[A]TNTGA |
| | | | GTGGTCAATCTTGATGGAAAGAAATGCTTAGGGAT |
| | | | GGTGTTTGTNTGCTAAGAATGGAGAAGAAACCAAT |
| | | | AAGCTAAGATCAGTTCATGTCTCGT |
| 16 | 8 | Chr4_48338565 | AACGGTTGGAATTTTTCTGAAGACATTACTGTTTTG |
| | | | TAATTGTCCATGAAGGTGACTAGCTTCGGGAATGG |
| | | | TCATTTTAGGTGCAAGCGATGATCTTGTT[C]TNTGA |
| | | | GTGGTCAATCTTGATGGAAAGAAATGCTTAGGGAT |
| | | | GGTGTTTGTNTGCTAAGAATGGAGAAGAAACCAAT |
| | | | AAGCTAAGATCAGTTCATGTCTCGT |
| 17 | 9 | Chr4_52795726 | CTATTAGTTTTGATGTGTCATTTTATGGTGCTGATTT |
| | | | TGTTTATGGTATTCCTGAACATGCTACTAGTCTTGC |
| | | | ACTTAAGCCTACTAGAGGCCCCGGTAT[C]GAACAT |
| | | | TCGGAACCTTATAGGCTTTTTAACTTGGATGTGTTT |
| | | | GAATATCTTCATGAGTCGCCTTTCGGGTTGTATGGA |
| | | | TCNATTCCGTTCATGCTTGGTC |
| 18 | 9 | Chr4_52795726 | CTATTAGTTTTGATGTGTCATTTTATGGTGCTGATTT |
| | | | TGTTTATGGTATTCCTGAACATGCTACTAGTCTTGC |
| | | | ACTTAAGCCTACTAGAGGCCCCGGTAT[T]GAACAT |
| | | | TCGGAACCTTATAGGCTTTTTAACTTGGATGTGTTT |
| | | | GAATATCTTCATGAGTCGCCTTTCGGGTTGTATGGA |
| | | | TCNATTCCGTTCATGCTTGGTC |
| 19 | 10 | Chr4_55593004 | TGCGCCAAATTATCAACCTGTGGTGCATGAACTTAT |
| | | | ACATGAAAAAACAAAACGAACTCATACATGAAAA |
| | | | CCAAAAGAGGTGGACCCTAAATGTAATAAC[A]AAC |
| | | | CTGGTGAGTGAGCCACATGAGCAAGATATCAACAG |
| | | | CAGGGACCAAGACACTTGAAGAAGCATCACCATCA |
| | | | ACATCCCCATCATCTTGTTGGATTACC |
| 20 | 10 | Chr4_55593004 | TGCGCCAAATTATCAACCTGTGGTGCATGAACTTAT |
| | | | ACATGAAAAAACAAAACGAACTCATACATGAAAA |
| | | | CCAAAAGAGGTGGACCCTAAATGTAATAAC[T]AAC |
| | | | CTGGTGAGTGAGCCACATGAGCAAGATATCAACAG |
| | | | CAGGGACCAAGACACTTGAAGAAGCATCACCATCA |
| | | | ACATCCCCATCATCTTGTTGGATTACC |
| 21 | 11 | Chr4_55597074 | AGAATGTGAGGGGAAGAAACMGAAGATGCATTGC |
| | | | ATGAAGAAGGATGRAGTATAATAGAACCCCAATCC |
| | | | CTCTCTCCATACTTGGCCTCTTTCAGTCTTC[T]CCTT |
| | | | ACTAACAATCCTCCCACATCTTCTTTTCTCATCTCA |
| | | | GAAACACTCCTCAGTATTGCAAGGCAYAATAACAG |
| | | | CACGAAAaCAGCRTCTTCTTCTGGC |
| 22 | 11 | Chr4_55597074 | AGAATGTGAGGGGAAGAAACMGAAGATGCATTGC |
| | | | ATGAAGAAGGATGRAGTATAATAGAACCCCAATCC |
| | | | CTCTCTCCATACTTGGCCTCTTTCAGTCTTC[G]CCTT |
| | | | ACTAACAATCCTCCCACATCTTCTTTTCTCATCTCA |
| | | | GAAACACTCCTCAGTATTGCAAGGCAYAATAACAG |
| | | | CACGAAAaCAGCRTCTTCTTCTGGC |
| 23 | 12 | Chr9_21546628 | AAAGAAATACTTTGACATGGTGGCGTAARTATTTTC |
| | | | CCGTGCTTAGACTGATAAATCCAACATCAATTCTAT |
| | | | TATCATGAAAAAGCAAATTTACCTCTTT[G]GATATT |
| | | | TGCTGCAGAATAGGTATGATCTCAGCGAAGTCAGG |
| | | | TCTCATTGCAGGATCTTGCTGCCAGCATCTTTCAAG |
| | | | AAGCTCGGTAAGCTTGGGATGAC |
| 24 | 12 | Chr9_21546628 | AAAGAAATACTTTGACATGGTGGCGTAARTATTTTC |
| | | | CCGTGCTTAGACTGATAAATCCAACATCAATTCTAT |
| | | | TATCATGAAAAAGCAAATTTACCTCTTT[T]GATATT |
| | | | TGCTGCAGAATAGGTATGATCTCAGCGAAGTCAGG |
| | | | TCTCATTGCAGGATCTTGCTGCCAGCATCTTTCAAG |
| | | | AAGCTCGGTAAGCTTGGGATGAC |
| 25 | 13 | Chr9_21859451 | AAGCAGATCCTTAACTTCCCTTCTTAACTGACGCAG |
| | | | TTTTGAGTCTTAAGAATGACAACCCTATGCTTTCAT |
| | | | TTGGATATTCAATGTCTACTGAAGCCAC[T]ACTACA |
| | | | GGCTTGATTACTGTTAAATTTGTACCAAAATGGGTA |
| | | | AATTACAGTTTATTGTTCTACTGATAATGATTTTTG |
| | | | TAAACATTCAAGTGCTTTGTAT |
| 26 | 13 | Chr9_21859451 | AAGCAGATCCTTAACTTCCCTTCTTAACTGACGCAG |
| | | | TTTTGAGTCTTAAGAATGACAACCCTATGCTTTCAT |
| | | | TTGGATATTCAATGTCTACTGAAGCCAC[C]ACTACA |
| | | | GGCTTGATTACTGTTAAATTTGTACCAAAATGGGTA |
| | | | AATTACAGTTTATTGTTCTACTGATAATGATTTTTG |
| | | | TAAACATTCAAGTGCTTTGTAT |
| 27 | 14 | Chr9_22691063 | CAAAAGACTATAAGGGTCAAGTTGAAGCAAACTTG |
| | | | AGAGTGGTCCTCCACTGGATCATTTGTCTCTATTGT |
| | | | CTTAATGCAACAAACGACTTGAAACCATC[T]AAAA |
| | | | ACAATAGATAGGATGATTCCACTAGAATCATCATT |
| | | | GCGAATAAGCAACCAAGGATCTTTATTGTTTACCA |
| | | | CATAAACATTCATATTGATCACCATG |
| 28 | 14 | Chr9_22691063 | CAAAAGACTATAAGGGTCAAGTTGAAGCAAACTTG |
| | | | AGAGTGGTCCTCCACTGGATCATTTGTCTCTATTGT |
| | | | CTTAATGCAACAAACGACTTGAAACCATC[C]AAAA |
| | | | ACAATAGATAGGATGATTCCACTAGAATCATCATT |
| | | | GCGAATAAGCAACCAAGGATCTTTATTGTTTACCA |
| | | | CATAAACATTCATATTGATCACCATG |
| 29 | 15 | Chr9_23659048 | ATATCATTGAAAAAATAATATTGAGGAAATAAAGT |
| | | | TCAACCTGTTTAGCACCTAGACCAAAGGTTGCATTG |
| | | | CCAGATAGATAAGGTTTCGAGCGATCAGC[G]GTTT |
| | | | TGCAGAGCATAGAAAGTGCTGCAGTAGCTCCCACT |
| | | | TGTATAGCCTGCATGTCCTCCAAAATGGTGAGAAG |
| | | | CTGAATAGCTTAAGATTCATAATTCT |
| 30 | 15 | Chr9_23659048 | ATATCATTGAAAAAATAATATTGAGGAAATAAAGT |
| | | | TCAACCTGTTTAGCACCTAGACCAAAGGTTGCATTG |
| | | | CCAGATAGATAAGGTTTCGAGCGATCAGC[A]GTTT |
| | | | TGCAGAGCATAGAAAGTGCTGCAGTAGCTCCCACT |
| | | | TGTATAGCCTGCATGTCCTCCAAAATGGTGAGAAG |
| | | | CTGAATAGCTTAAGATTCATAATTCT |
| 31 | 16 | Chr9_24117497 | AGGTAATATATAAAACAGAGGCAATCTCACAATAA |
| | | | AACAGTTATCATGGSGATCAGATTCACAAACCCGT |
| | | | AACAGCAACATGAGGGTCAAAACAGCCACC[A]GAT |
| | | | AAACTTCAAAAAGCCCGACCCGACCCCAACTACTG |
| | | | GGCAGCAACAGCCAACTTAAAGCTTACCAGCCCAA |
| | | | GCTACGAYCAATCAACATKGTAACATC |
| 32 | 16 | Chr9_24117497 | AGGTAATATATAAAACAGAGGCAATCTCACAATAA |
| | | | AACAGTTATCATGGSGATCAGATTCACAAACCCGT |
| | | | AACAGCAACATGAGGGTCAAAACAGCCACC[G]GA |
| | | | TAAACTTCAAAAAGCCCGACCCGACCCCAACTACT |
| | | | GGGCAGCAACAGCCAACTTAAAGCTTACCAGCCCA |
| | | | AGCTACGAYCAATCAACATKGTAACATC |
| 33 | 17 | Chr9_24118744 | GACACCACCTAATCAAATAATGCTTTCTTCATTAGT |
| | | | AGGGAACCCTATCCAAGAACAAGAACTTACCTCAA |
| | | | GTGATTTTCGAGGTGAGGGGCTACGAGAG[A]TTCC |
| | | | AGCTTCCCCACGCGGAGAAACYGCCTGRGGAGAAG |
| | | | CCAAACGATGAGGAATTGGTGCWCGTTCATCRAGG |
| | | | CCGYGCTTCTCTGGGCTACGACTCCT |
| 34 | 17 | Chr9_24118744 | GACACCACCTAATCAAATAATGCTTTCTTCATTAGT |
| | | | AGGGAACCCTATCCAAGAACAAGAACTTACCTCAA |
| | | | GTGATTTTCGAGGTGAGGGGCTACGAGAG[G]TTCC |
| | | | AGCTTCCCCACGCGGAGAAACYGCCTGRGGAGAAG |
| | | | CCAAACGATGAGGAATTGGTGCWCGTTCATCRAGG |
| | | | CCGYGCTTCTCTGGGCTACGACTCCT |
| 35 | 18 | Chr9_24213487 | AAGAATTCTTTGTGTTAAGGTGGTTGTGTGATGTAT |
| | | | GTTTGATAGTAGCAAAGATTCCTTTGATATATGTAG |
| | | | ATTAATATAGTCAATTTTTATCTATCAT[C]TGTAGT |
| | | | AGCACTCTCAAGTTCATCTGATATTCGTGTAATAAT |
| | | | TCTGAACGCTTGAATGCATTGGTTGTTTTTGTTTAG |
| | | | ACATTAAACTCTTTTGTTCAGG |
| 36 | 18 | Chr9_24213487 | AAGAATTCTTTGTGTTAAGGTGGTTGTGTGATGTAT |
| | | | GTTTGATAGTAGCAAAGATTCCTTTGATATATGTAG |
| | | | ATTAATATAGTCAATTTTTATCTATCAT[T]TGTAGT |
| | | | AGCACTCTCAAGTTCATCTGATATTCGTGTAATAAT |
| | | | TCTGAACGCTTGAATGCATTGGTTGTTTTTGTTTAG |
| | | | ACATTAAACTCTTTTGTTCAGG |
| 37 | 19 | Chr9_24369620 | AACTTATATTACATGAGTGACATTCATATAGGTCAC |
| | | | TTACGCGAACATCTGGTAGATTATCAATGCCTAAAT |
| | | | TTGAAGTATCCTTCTCATATTCAACAGG[T]CCACAT |
| | | | GAATTACTTATAGTTGCAAGGGAAAGGTCCAGCGT |
| | | | GGAGTTGCCATCCTCAGAATTCACGCACATATGCT |
| | | | GTTAGATAGCAGCATACAAATGTT |
| 38 | 19 | Chr9_24369620 | AACTTATATTACATGAGTGACATTCATATAGGTCAC |
| | | | TTACGCGAACATCTGGTAGATTATCAATGCCTAAAT |
| | | | TTGAAGTATCCTTCTCATATTCAACAGG[C]CCACAT |
| | | | GAATTACTTATAGTTGCAAGGGAAAGGTCCAGCGT |
| | | | GGAGTTGCCATCCTCAGAATTCACGCACATATGCT |
| | | | GTTAGATAGCAGCATACAAATGTT |

1. A Cercospora-resistant Beta vulgaris plant comprising a first Cercospora resistance providing genomic fragment on chromosome 4, wherein said first Cercospora resistance providing genomic fragment comprises at least one sequence from the group consisting of SEQ ID Nos. 1, 3, 5, 7, 9, 11, 13, and 15.

2. The Beta vulgaris plant according to claim 1, wherein said first Cercospora resistance providing genomic fragment further comprises at least one sequence selected from the group consisting of SEQ ID Nos. 17, 19, and 21.

3. The Beta vulgaris plant according to claim 1, wherein said plant comprises a second Cercospora resistance providing genomic fragment on chromosome 9, wherein said second Cercospora resistance providing genomic fragment comprises at least one sequence selected from the group consisting of SEQ ID Nos. 23, 25, 27, 29, 31, 33, 35, and 37.

4. The Beta vulgaris plant according to claim 1, wherein the first and second Cercospora resistance providing genomic fragments are obtained, obtainable, or are from a Beta vulgaris plant deposited under deposit accession number NCIMB 43769.

5. The Beta vulgaris plant according to claim 1, comprising a first Cercospora resistance providing genomic fragment on chromosome 4, wherein said first Cercospora resistance providing genomic fragment comprises SEQ ID Nos. 1, 3, 5, 7, 9, 11, 13, and 15, and a second Cercospora resistance providing genomic fragment on chromosome 9, wherein said second Cercospora resistance providing genomic fragment comprises SEQ ID Nos. 23, 25, 27, 29, 31, 33, 35, and 37.

6. The Beta vulgaris plant according to claim 1, wherein said plant is cytoplasmic male sterile (CMS).

7. The Beta vulgaris plant according to claim 1, wherein said plant is a hybrid plant.

8. The Beta vulgaris plant according to claim 1, wherein said plant is a Beta vulgaris plant deposited under accession number NCIMB 43769.

9. The Beta vulgaris plant according to claim 1, wherein the plant is selected from the group consisting of Beta vulgaris ssp. vulgaris var. conditiva, Beta vulgaris ssp. vulgaris var. altissima, Beta vulgaris subsp. vulgaris var. flavescens, Beta vulgaris subsp. vulgaris var. cicla, and Beta vulgaris ssp. vulgaris var. crassa.

10. A method for identifying a genomically encoded resistance against the plant pathogen Cercospora as found in a Beta vulgaris plant deposited under accession number NCIMB 43769, the method comprises the step of identifying a first Cercospora resistance providing genomic fragment on chromosome 4 by detecting at least one genomic sequence selected from the group consisting of SEQ ID Nos. 1, 3, 5, 7, 9, 11, 13, 15, 17, 19, and 21.

11. M xl according to claim 10, wherein the method further comprises identifying a second Cercospora resistance providing genomic fragment located on chromosome 9 by detecting at least one genomic sequence selected text from the group consisting of SEQ ID Nos. 23, 25, 27, 29, 31, 33, 35, and 37.

12. A method for providing a Beta vulgaris plant according to claim 1, comprising steps of obtaining Cercospora susceptible plant material from a plant of the species Beta vulgaris, subjecting said Cercospora susceptible plant material to mutagenesis to create mutagenized plant material, and analyzing said mutagenized plant material to identify a plant having resistance to Cercospora beticola.

13. A seed or plant part of a Beta vulgaris plant according to claim 1.

14. The seed according to claim 13 wherein said seed is polished, coated, encrusted, pelleted, or primed.

15. A molecular marker selected from the group consisting of SEQ ID Nos. 1, 3, 5, 7,
9, 11, 13, 15, 17, 19, 21, 23, 25, 27, 29, 31, 33, 35, and 37.
